SEQUENCE LISTING (4 sequences)

SEQ ID NO: 1 - DNA, 18 bp, Artificial Sequence
  Synthetic Oligonucleotide
ggcctcggcctcctgtat

SEQ ID NO: 2 - DNA, 19 bp, Artificial Sequence
  Synthetic Oligonucleotide
ccacttccccttgtgcttt

SEQ ID NO: 3 - DNA, 22 bp, Artificial Sequence
  Synthetic Oligonucleotide
cggcctcggcctcctgtatata

SEQ ID NO: 4 - DNA, 22 bp, Artificial Sequence
  Synthetic Oligonucleotide
tccacttccccttgtgctttgt

(19) United States
(12) Patent Application Publication
(10) Pub. No.: US 2016/0002743 A1
(43) Pub. Date: Jan. 7, 2016

(54) HIGH RESOLUTION MELTING ANALYSIS ASSAY FOR THE DETECTION OF VIRAL DNA

(71) Applicant: BIOGEN MA INC., Cambridge, MA (US)

(72) Inventors: Soma Ray, Needham, MA (US); Ian David Faulkner, Boston, MA (US)

(73) Assignee: Biogen MA Inc., Cambridge, MA (US)

(21) Appl. No.: 14/775,797

(22) PCT Filed: Mar. 15, 2014
(86) PCT No.: PCT/US14/29967

§ 371 Date: Sep. 14, 2015

Related U.S. Application Data
(60) Provisional application No. 61/792,479, filed on Mar. 15, 2013.

(51) Int. Cl. C12Q 1/70

(52) U.S. Cl. CPC C12Q 1/701

(57) ABSTRACT

In one aspect, the disclosure provides methods, kits and compositions for determining the presence of a JC virus mutant in a sample.

### RELATED APPLICATIONS

This application claims the benefit under 35 U.S.C. §119(e) of U.S. provisional application No. 61/792,479, filed Mar. 15, 2013, which is incorporated by reference herein in its entirety.

### FIELD OF THE INVENTION

The invention is in the field of detection of nucleic acids in biological samples.

### BACKGROUND OF THE INVENTION

JC virus (JCV) is a human polyomavirus known to cause a rare disorder of the central nervous system (CNS) called progressive multifocal leukoencephalopathy (PML). The detection of JCV and JCV mutants in biological samples is challenging. Thus, improved methods for the detection of JCV and JCV mutants in biological samples are needed.

### SUMMARY OF THE INVENTION

In some aspects, the present disclosure provides methods, kits and compositions for determining the presence of a JC virus (JCV) mutant in a sample.

In some aspects, the present disclosure provides methods for determining the presence of a JCV mutant in a sample, the methods comprising: amplifying nucleic acid of a JCV in a sample in the presence of a dye that preferentially binds double-stranded nucleic acid over single stranded nucleic acid, changing the temperature of the sample and monitoring a signal corresponding to the dye binding to double-stranded nucleic acid to determine the melting temperature of double-stranded nucleic acid in the sample, and identifying the sample as comprising a JCV mutant if the melting temperature observed for the sample is different from the melting temperature of a control sample comprising double-stranded JCV nucleic acid of a non-mutant JCV.

In some embodiments of the present disclosure, amplifying nucleic acid of a JCV comprises contacting the sample comprising nucleic acid of a JCV with at least two primers that can hybridize to the nucleic acid of a JCV.

In some aspects, the present disclosure provides methods for determining the presence of a JCV mutant in a sample, the methods comprising obtaining a sample comprising double-stranded JCV nucleic acid and a dye that preferentially binds double-stranded nucleic acid over single stranded nucleic acid, changing the temperature of the sample and monitoring a signal corresponding to the dye binding to double-stranded nucleic acid to determine the melting temperature of double-stranded nucleic acid in the sample, and identifying the sample as comprising a JCV mutant if the melting temperature observed for the sample is different from the melting temperature of a control sample comprising double-stranded JCV nucleic acid of a non-mutant JCV.

In some embodiments of the present disclosure, a double-stranded JCV nucleic acid is amplified.

In some embodiments of the present disclosure, changing the temperature of the sample comprises heating the sample.

In some embodiments of the present disclosure, a sample is a biological sample.

In some embodiments of the present disclosure, a sample is from a subject suspected of being infected with JCV.

In some embodiments of the present disclosure, a sample is a blood sample.

In some embodiments of the present disclosure, a sample is a cerebrospinal fluid (CSF) sample.

In some embodiments of the present disclosure, a sample is a urine sample.

In some embodiments of the present disclosure, less than 10,000 copies of JCV are present in a sample.

In some embodiments of the present disclosure, less than 1,000 copies of JCV are present in a sample.

In some embodiments of the present disclosure, less than 100 copies of JCV are present in a sample.

In some embodiments of the present disclosure, a double-stranded JCV nucleic acid comprises a noncoding control region (NCCR) of the JCV.

In some embodiments of the present disclosure, a double-stranded JCV nucleic acid is a part of a NCCR of the JCV.

In some embodiments of the present disclosure, a double-stranded JCV nucleic acid is at least 50 nucleotides in length.

In some embodiments of the present disclosure, a double-stranded JCV nucleic acid is at least 100 nucleotides in length.

In some embodiments of the present disclosure, a double-stranded JCV nucleic acid is at least 500 nucleotides in length.

In some embodiments of the present disclosure, a mutation includes less than 20 nucleotides.

In some embodiments of the present disclosure, a mutation includes less than 10 nucleotides.

In some embodiments of the present disclosure, a mutation is a single base pair mutation.

In some embodiments of the present disclosure, a primer is a nucleic acid primer.

In some embodiments of the present disclosure, a primer has a sequence comprising SEQ ID NO:1 or SEQ ID NO:2. In some embodiments of the present disclosure, a primer has a sequence comprising SEQ ID NO:3 or SEQ ID NO:4.

In some embodiments of the present disclosure, a dye is an intercalating dye.

In some embodiments of the present disclosure, a dye is a fluorescent dye.

In some embodiments of the present disclosure, a dye is a SYBR Green I dye.

In some embodiments of the present disclosure, a sample is from a subject, and if the sample is identified as comprising a JCV mutant, the subject is identified as being at risk for developing progressive multifocal leukoencephalopathy (PML).

In some embodiments of the present disclosure, a sample is from a subject, and if the sample is identified as comprising a JCV mutant, the subject is identified as being inappropriate for treatment comprising one or more immunosuppressants. In some embodiments, treatment comprising one or more immunosuppressants comprises treatment including natalizumab.

In some embodiments of the present disclosure, a sample is from a subject, and if the sample is identified as comprising a JCV mutant, and the subject is receiving treatment comprising one or more immunosuppressants, the subject is identified as requiring adjustment or termination of treatment comprising one or more immunosuppressants. In some embodiments, treatment comprising one or more immunosuppressants comprises treatment including natalizumab.

In one aspect, the disclosure provides a kit comprising an intercalating fluorescent dye and double-stranded JCV nucleic acid of a non-mutant JCV.

In some embodiments of the present disclosure, a kit further comprises a first nucleic acid primer having a sequence comprising SEQ ID NO:1 and a second nucleic acid primer having a sequence comprising SEQ ID NO:2. In some embodiments of the kits provided herein, a kit further comprises a first nucleic acid primer having a sequence comprising SEQ ID NO:3 and a second nucleic acid primer having a sequence comprising SEQ ID NO:4.

In some aspects, the present disclosure provides a nucleic acid primer having a sequence comprising SEQ ID NO:1.

In some aspects, the present disclosure provides a nucleic acid primer having a sequence comprising SEQ ID NO:2.

In some aspects, the present disclosure provides a nucleic acid primer having a sequence comprising SEQ ID NO:3.

In some aspects, the present disclosure provides a nucleic acid primer having a sequence comprising SEQ ID NO:4.

These and other aspects of the present disclosure are described in more detail herein.

### BRIEF DESCRIPTION OF THE DRAWINGS

The figures are illustrative only and are not required for enablement of the invention disclosed herein.

FIG. 1 shows an overview of an example of a high resolution melt (HRM) curve analysis.

FIG. 2 shows an example of noncoding control region (NCCR) sequence rearrangements in JC virus (FIG. 2A illustrates sequence blocks that are frequently duplicated or deleted, FIG. 2B illustrates examples of specific sequence alterations).

FIG. 3 shows that a HRM curve analysis can differentiate Mad-1 from archetype.

FIG. 4 shows that a HRM curve analysis can differentiate a range of mutations on the NCCR of JC virus derived from TYSABRI®-treated PML patients.

FIG. 5 shows that a HRM curve analysis can differentiate mixtures of different NCCRs (e.g., MAD-1 in presence of Archetype NCCR).

FIG. 6 shows that HRM curves are reproducible across dilutions of at least 6 orders of magnitude.

FIG. 7 shows different HRM signatures for different NCCR rearrangements in clinical samples.

### DETAILED DESCRIPTION OF THE INVENTION

Provided herein, in some aspects, are methods, compositions and kits that permit detection of John Cunningham virus (JCV) mutant nucleic acid in a sample (e.g., biological sample).

In some embodiments, the disclosure relates to detecting JCV mutants in a patient sample in order to evaluate the risk of progressive multifocal leukoencephalopathy (PML) in a patient. Although primary infection with JCV often occurs asymptomatically during childhood, JCV is typically disseminated throughout the body, possibly through viraemia. While infection by JCV is asymptomatic in most subjects, infection may result in serious conditions (e.g., PML) and even death. Subjects most susceptible to PML are subjects who are immuno-compromised (e.g., AIDS patients) or who are undergoing treatment with immuno-suppressants, for instance, after organ transplant or to treat an inflammation related condition, such as multiple sclerosis (e.g., using natalizumab or other immunosuppressive drug).

Within individual hosts, JCV can persist in two (or more) forms: a latent, nonpathogenic form and a virulent neurotropic form (see e.g., Reid et al., Journal of Infectious Diseases 2011, 204:237-244). The neurotropic form often contains mutations, for instance, in the noncoding control region (NCCR) of JCV, and is typically found in the cerebrospinal fluid (CSF), brain or blood of PML patients. A nonpathogenic form of JCV is most frequently detected in urine, and its NCCR generally is not rearranged. In one aspect, the disclosure provides methods for determining if a biological sample (e.g., CSF or blood) from a subject comprises a JCV with mutations in the NCCR, which may help diagnose if the subject has the latent, nonpathogenic form or the virulent neurotropic form of JCV.

In one aspect, the disclosure provides a method for determining the presence of a JCV mutant in a sample, the method comprising amplifying nucleic acid of a JCV in a sample in the presence of a dye that preferentially binds double-stranded nucleic acid over single stranded nucleic acid, changing the temperature of the sample and monitoring a signal corresponding to the dye binding to double-stranded nucleic acid to determine the melting temperature of double-stranded nucleic acid in the sample, and identifying the sample as comprising a JCV mutant if the melting temperature observed for the sample is different from the melting temperature of a control sample comprising double-stranded JCV nucleic acid of a non-mutant JCV. In some embodiments, changing the temperature of the sample comprises heating the sample.

In one aspect, the disclosure provides a method for determining the presence of a JCV mutant in a sample, the method comprising obtaining a sample comprising double-stranded JCV nucleic acid and a dye that preferentially binds double-stranded nucleic acid over single stranded nucleic acid, changing the temperature of the sample and monitoring a signal corresponding to the dye binding to double-stranded nucleic acid to determine the melting temperature of double-stranded nucleic acid in the sample, and identifying the sample as comprising a JCV mutant if the melting temperature observed for the sample is different from the melting temperature of a control sample comprising double-stranded JCV nucleic acid of a non-mutant JCV. In some embodiments, changing the temperature of the sample comprises heating the sample.

Methods for determining the presence of a JCV mutant in a sample provided herein are based on differences in melting temperature between a double-stranded nucleic acid suspected of containing a mutation (e.g., single nucleotide substitution) and a corresponding wild-type double-stranded nucleic acid. Generally, when comparing two double-stranded nucleic acids of the same length, the double-stranded nucleic acid with a higher guanine-cytosine (GC) content (greater number of GC nucleic acid base pairs) will have a higher melting temperature. A GC nucleotide base pair melts (dissociates) at a higher temperature relative to an adenine-thymine (AT) nucleotide base pair. The difference in melting temperature between a GC nucleic acid base pair and an AT nucleic acid base pair is due to an additional hydrogen bond in the GC nucleic acid base pair relative to the AT nucleic acid base pair. For instance, a wild-type (non-mutant) double-stranded nucleic acid with a GC nucleic acid base pair at position N will have a higher melting temperature than a corresponding mutant nucleic acid with an AT base pair at position N. Conversely, a wild-type (non-mutant) double-stranded nucleic acid with an AT nucleic acid base pair at position N will have a lower melting temperature than a corresponding mutant nucleic acid with a GC base pair at position N. In addition, nucleic acids having different lengths can have different melting temperatures, with longer double-stranded nucleic acids typically having higher melting temperatures than corresponding shorter double-stranded nucleic acids. Accordingly, deletions or duplications in a nucleic acid suspected of containing a mutation also can impact the melting temperature of a nucleic acid.

In one aspect, if the melting temperature of a wild type double-stranded nucleic acid is known, or can readily be determined (e.g., based on length and nucleotide content), a nucleic acid suspected of having a mutation, referred to as “a suspected mutant nucleic acid,” can be tested by comparing the melting temperature of the suspected mutant nucleic acid to the melting temperature of the corresponding wild-type nucleic acid. If the melting temperature of the suspected mutant nucleic acid is different from the corresponding wild-type nucleic acid, it can be inferred that the suspected mutant nucleic acid has a GC to AT mutation (or an AT to GC mutation). For example, if the melting temperature (Tm) of the suspected mutant nucleic acid is lower than the Tm of the corresponding wild-type nucleic acid, one can infer that the suspected mutant nucleic acid has a G to A, G to T, C to A, or C to T mutation. Alternatively, if the Tm of the suspected mutant nucleic acid is higher than the Tm of the corresponding wild-type nucleic acid, one can infer that the suspected mutant nucleic acid has an A to G, T to G, A to C, or T to C mutation.

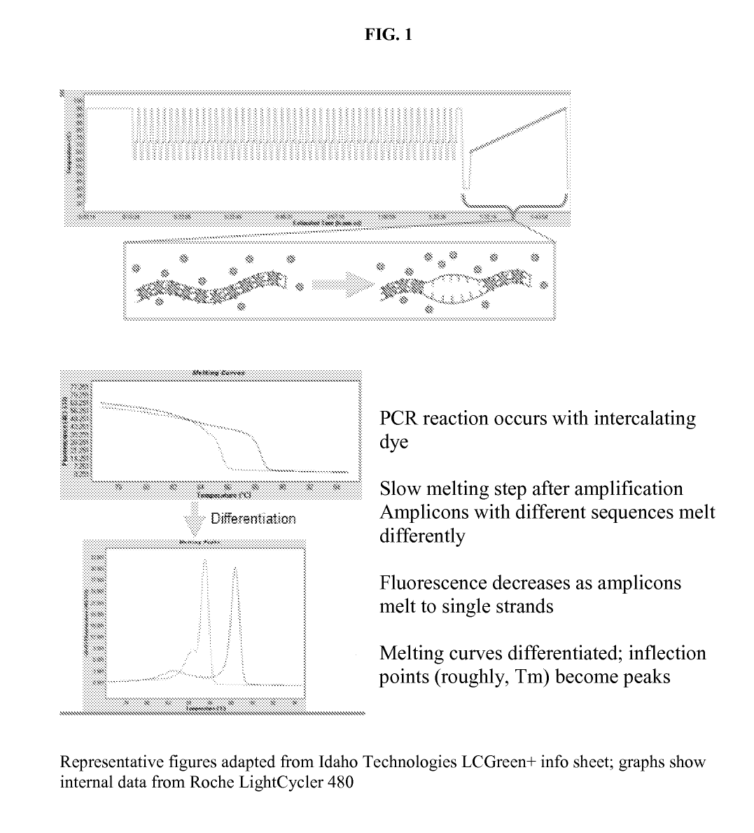

This physical phenomenon (the difference in melting temperatures) is employed in high resolution melting (HRM) (see e.g., FIG. 1). In HRM, a double-stranded nucleic acid is incubated with an agent that preferentially binds double-stranded nucleic acid and emits a signal if it binds double-stranded nucleic acid. An example of an agent that preferentially binds double-stranded nucleic acid is an intercalating dye. Examples of intercalating dyes are provided herein. Heating double-stranded nucleic acids results in a loss of, or decrease in, signal from the intercalating dye at the time of melting of the nucleic acid because the intercalating dye is no longer bound to the double-stranded nucleic acid. As described above, the melting temperature will depend on the length and nucleotide content of the double-stranded nucleic acid. Differences in melting temperature among double-stranded nucleic acids of the same length typically correspond to differences in nucleotide content. It should be appreciated that double-stranded nucleic acids of the same length with the same melting temperature generally have the same nucleotide content. If a sample is evaluated for the presence of a JCV mutant nucleic acid, and the melting temperature of double-stranded JCV nucleic acid from the sample has the same melting temperature as a non-mutant, or wild-type, JCV double-stranded nucleic acid, it can be inferred that the sample contains a non-mutant, or wild-type, JCV nucleic acid.

However, it should be appreciated that a long double-stranded nucleic acid molecule can be so stable that mutations (including certain substitutions, deletions, insertions, or duplications) do not have a significant impact on the overall melting temperature of the nucleic acid, making it difficult to detect or infer the presence of the mutation based on the melting temperature alone.

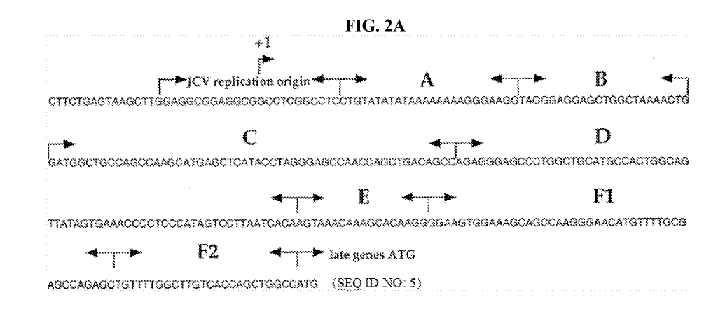

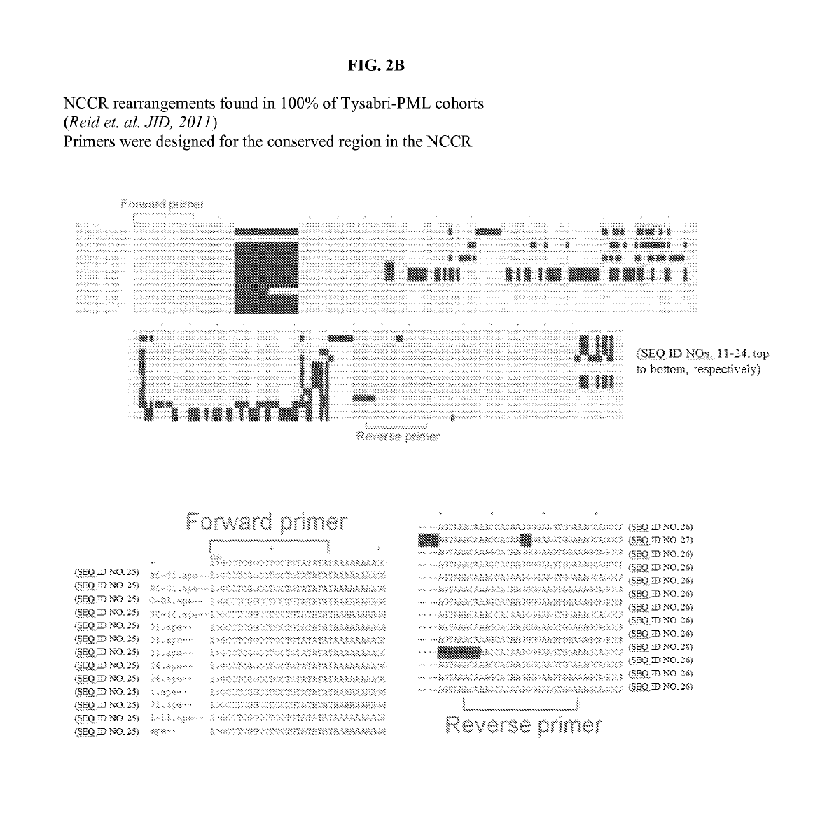

Surprisingly, certain JCV nucleic acid regions described herein can be assayed using melting techniques (e.g., HRM techniques) described herein to detect or infer the presence of mutations associated with JCV pathogenicity. FIG. 2A illustrates certain blocks of sequence in the JCV NCCR region (SEQ ID NO: 5) that can be expanded (e.g., due to duplication or insertion) or deleted or that can contain expansions or deletions in JCV associated with pathogenicity. Surprisingly, techniques described herein can be used to detect or infer expansions or deletions within these regions by assaying biological samples without sequencing the JCV nucleic acid.

Accordingly, methods and compositions described herein can be used to detect or infer the presence of mutations within a JCV nucleic acid region that can be indicative of a risk for JCV pathogenicity. It also should be appreciated that methods and compositions described herein can be used to determine or infer that a JCV nucleic acid appears to be normal (e.g., wild-type or archetype) in that it does not have properties indicative of the presence of certain mutations associated with JCV pathogenicity.

Thus, in one aspect, the disclosure provides methods, kits and compositions for determining the presence of wild-type JCV in a sample. In some embodiments, the disclosure provides methods, kits and compositions for determining the presence of a JCV mutant and wild-type JCV in a sample.

It should be appreciated that wild-type JCV can have more than one nucleotide sequence. That is, a wild-type JCV sequence can vary. For instance, JCV has several wild-type, or “base” strains. Thus, mutations in JCV nucleic acid detected by HRM may be compared to multiple base strains, which can be “traditional” wild-type strains (e.g., referred to as archetype strains that are common non-pathogenic strains that can differ from each other by, for example, 1 to 3 single nucleotide polymorphisms or, in some instances, up to 11 contiguous nucleotide deletions. The wild-type, or non-mutant, strain to which the suspected mutant JCV sequences are compared may depend on the application (e.g., when analyzing HIV positive vs. HIV negative subjects), or may depend on the geographical origin of the subject (e.g., Asia vs. Europe).

A non-limiting example of an archetype sequence of the NCCR region that can be used as a reference includes SEQ ID NO: 6:

GCCTCGGCCTCCTGTATATATAAAAAAAAGGGAAGGTAGGGAGGAGCTGG
CTAAAACTGGATGGCTGCCAGCCAAGCATGAGCTCATACCTAGGGAGCCA
ACCAGCTGACAGCCAGAGGGAGCCCTGGCTGCATGCCACTGGCAGTTATA
GTGAAACCCCTCCCATAGTCCTTAATCACAAGTAAACAAAGCACAAGGGG
AAGTGGAAAGCAGCCAAGGGAACATGTTTTGCGAGCCAGAGCTGTTTTGG
CTTGTCACCAGCTGGCCATG

In some embodiments, a subregion of SEQ ID NO: 6 is used (4 bases are trimmed from the 5′ end of SEQ ID NO: 6, and a longer region is trimmed from the 3′ end of SEQ ID NO: 6:

CGGCCTCGGCCTCCTGTATATATAAAAAAAAGGGAAGGTAGGGAGGAGCT
GGCTAAAACTGGATGGCTGCCAGCCAAGCATGAGCTCATACCTAGGGAGC
CAACCAGCTGACAGCCAGAGGGAGCCCTGGCTGCATGCCACTGGCAGTTA
TAGTGAAACCCCTCCCATAGTCCTTAATCACAAGTAAACAAAGCACAAGG
GGAAGTGGA

In some embodiments, primers are used to amplify this trimmed subregion of SEQ ID NO: 6 (e.g., primers having sequences of SEQ ID NO: 3 and SEQ ID NO: 4 can be used to amplify this region to use it as a reference in HRM methods described herein).

Other non-limiting examples of archetype sequences that can be used as references in HRM methods described herein include sequences that differ from SEQ ID NO: 6 by one or a few changes as described below:

SEQ ID NO: 7 includes a common SNP (A to C at position 134):

GGCCTCCTGTATATATAAAAAAAAGGGAAGGTAGGGAGGAGCTGGCTAAA
ACTGGATGGCTGCCAGCCAAGCATGAGCTCATACCTAGGGAGCCAACCAG
CTGACAGCCAGAGGGAGCCCTGGCTGCCTGCCACTGGCAGTTATAGTGAA
ACCCCTCCCATAGTCCTTAATCACAAGTAAACAAAGCACAAGGGGAAGTG
GA

SEQ ID NO: 8 includes a common SNP (C to A at position 160):

GGCCTCGGCCTCCTGTATATATAAAAAAAAGGGAAGGTAGGGAGGAGCTG
GCTAAAACTGGATGGCTGCCAGCCAAGCATGAGCTCATACCTAGGGAGCC
AACCAGCTGACAGCCAGAGGGAGCCCTGGCTGCATGCCACTGGCAGTTAT
AGTGAAACCACTCCCATAGTCCTTAATCACAAGTAAACAAAGCACAAGGG
GAAGTGGA

SEQ ID NO: 9 includes a common single nucleotide deletion (A at position 166):

GGCCTCGGCCTCCTGTATATATAAAAAAAAGGGAAGGTAGGGAGGAGCTG
GCTAAAACTGGATGGCTGCCAGCCAAGCATGAGCTCATACCTAGGGAGCC
AACCAGCTGACAGCCAGAGGGAGCCCTGGCTGCATGCCACTGGCAGTTAT
AGTGAAACCCCTCCCTAGTCCTTAATCACAAGTAAACAAAGCACAAGGGG
AAGTGGA

SEQ ID NO: 10 include a less common deletion (10 bp starting at position 48):

CCTCGGCCTCCTGTATATATAAAAAAAAGGGAAGGTAGGGAGGAGCTGGA
TGGCTGCCAGCCAAGCATGAGCTCATACCTAGGGAGCCAACCAGCTGACA
GCCAGAGGGAGCCCTGGCTGCCTGCCACTGGCAGTTATAGTGAAACCCCT
CCCATAGTCCTTAATCACAAGTAAACAAAGCACAAGGGGAAGTGGA

It should be appreciated that HRM has several limitation with respect to detecting differences in nucleotide content among double-stranded nucleic acid samples, and, thus, if differences in melting temperature between a wild-type JCV double-stranded nucleic acid and a suspected mutant JCV double-stranded nucleic acid are not detected, it may not be possible to infer (e.g., with certainty) the absence of a mutation in the suspected mutant JCV double-stranded nucleic acid. For example, many mutations are not detected by HRM. A single amino acid mutation from G to C (or C to G) or A to T (or T to A) will not result in change in melting temperature, and thus, such a mutation will not be detected by HRM analysis. Furthermore, some double mutations (e.g., G to T and T to G, or G to A and A to C) within a single nucleic acid will result in a mutant nuclei acid with the same melting temperature as the wild-type nucleic acid. Also, secondary structures formed in the nucleic acids to be analyzed, which may include non-traditional base pairing, may interfere with HRM measurements. Further, the specific nature of the mutations generally cannot be determined from the detected differences in melting temperature. Accordingly, an absence of a change in melting temperature can be used to infer that there are no mutations that are detected in an assay described herein. However, in some embodiments, one or more mutations may be present, despite an absence of a change in melting temperature.

Another limitation of HRM is related to the origin of the double-stranded nucleic acids to be analyzed. The nucleic acids have to be harvested from samples, often biological samples, and the nucleic acids generally have to be amplified before they can be analyzed by HRM. Amplification of nucleic acids may result in preferential amplification of one sequence over the other, and additional mutations that may be introduced though polymerase chain reaction (PCR).

Nonetheless, assays described herein are useful to evaluate the presence and/or risk of certain JCV mutations in a sample. For example, an absence of a change in melting temperature can be used to infer an absence of certain mutations as described herein, whereas the presence of a change in melting temperature can be used to infer the presence of one or more mutations that may represent an increased risk for JCV pathogenicity. It should be appreciated that in contrast to minor differences between archetype strains, certain mutations associated with JCV pathogenicity involve larger expansion or deletion of sequences (e.g., block sequences) within the NCCR region.

The limitations of using HRM to analyze samples for the presence of mutations are most acute in the analysis of viral mutations. Viruses are often found in very low numbers in cells or biological samples (e.g., blood and CSF), which makes obtaining representative samples of double-stranded virus nucleic acid for HRM analysis challenging.

Surprisingly, the methods provided herein permit detection of JCV mutant nucleic acid in a sample (e.g., biological sample). The results of the studies provided herein were unexpected, particularly in light of the size of the JCV genome, the nature of JCV mutations, the difficulties associated with obtaining representative samples of double-stranded JCV nucleic acid.

In one aspect, the disclosure provides a method for determining the presence of a JCV mutant in a sample. In one aspect, the disclosure provides a method for determining the presence of a JCV mutant in a sample, wherein less than 10,000 copies of the JCV mutant nucleic acid are present in the sample. In some embodiments of the methods provided herein, less than 1,000 copies of the JCV mutant nucleic acid are present in the sample. In some embodiments of the methods provided herein, less than 100 copies of the JCV mutant nucleic acid are present in the sample. In some embodiments of the methods provided herein, less than 10⁸, less than 10⁷, less than 10⁶, less than 10⁵ or less than 10⁴ copies of the JCV mutant nucleic acid are present in the sample.

In one aspect, the disclosure provides a method for determining the presence of a JCV mutant nucleic acid in a sample. In one aspect, the disclosure provides a method for determining the presence of a JCV mutant nucleic acid in a sample, wherein the JCV mutant nucleic acid contains a mutation. A “mutation” in a mutant nucleic acid, as used herein, refers to a change of nucleotide sequence in the mutant nucleic acid relative to a corresponding wild-type, or non-mutant, nucleic acid. In some embodiments, mutation includes a change of less than 20 nucleotides. In some embodiments of the methods provide herein, the mutation includes a change of less than 10 nucleotides. In some embodiments of the methods provide herein, the mutation is a single nucleotide change (e.g., resulting in a single base pair mutation). In some embodiments of the methods provide herein, the mutation includes a change of less than 100, less than 50, less than 40, less than 30, less than 20, less than 10, less than 5, or less than 2 nucleotides. A mutation may include a change of contiguous nucleotides or change of non-contiguous nucleotides. For example, in some embodiments, a mutation may include a change from ATTG (wild-type sequence) to CGGA (contiguous mutation sequence of 4 nucleotide changes, changes underlined) or a change from ATTG (wild-type sequence) to AGTA (non-contiguous mutation sequence of 2 nucleotide changes, changes underlined).

As discussed herein, a non-mutant or wild-type JCV nucleic acid can be a JCV nucleic acid that is known not to be associated with a high risk for PML. Accordingly, a mutation can be a risk factor, but because the sequence is not known, it is a factor that identifies a subject for further analysis. Thus, a mutant JCV nucleic acid that can be differentiated from a non-mutant (e.g., non-pathogenic archetype) nucleic acid by, for example, a difference in melting temperature as provided herein, in some embodiments, is subject to further analysis. In some embodiments, a mutant JCV nucleic acid, or a region of the mutant JCV nucleic acid suspected of containing a mutation that could be associated with increased risk for pathogenicity, is sequenced, thereby permitting identification of the particular mutation or mutations (e.g., one or more SNPs, expansions or deletions).

Mutations in JCV nucleic acids such as, for example, the NCCR region, are often random. FIG. 2B provides examples of certain known mutations associated with pathogenicity. However, these are non-limiting and in some embodiments other mutations in this region that are detectable using methods of the disclosure are risk factors for pathenogenicity. Thus, in some embodiments, mutations identified by methods provided herein will likely be novel and not previously associated with pathogenesis but, nonetheless, are potentially pathogenic. Therefore, subjects who are identified as having a novel mutation in a JCV nucleic acid, in some embodiment, are monitored more frequently than subjects with known JCV mutations (or more frequently relative to a subject with non-mutant or wild-type JCV nucleic acid. For example, a subject with a novel JCV mutation may be monitored for early diagnostic or clinical signs of pathogenesis (for example weekly, bi-weekly, monthly, every other month, every six months, or yearly). In some embodiments, the treatment of a subject (e.g., with a therapeutic drug, for example natalizumab, that can be immunosuppressive) is modified or terminated if one or more JCV mutations are detected according to methods described herein in a sample obtained from the subject.

### Intercalating Dye

In one aspect, the disclosure provides methods for determining the presence of a JCV or JCV mutant in a sample. In some embodiments, the methods include the use of a dye that preferentially binds double-stranded nucleic acid over single stranded nucleic acid. In some embodiments, the methods include the use of a dye that emits a signal when it binds double-stranded nucleic acid. In some embodiments, the dye is an intercalating dye. In some embodiments, the dye is a fluorescent dye. In some embodiments, the dye is a SYBR® Green (an asymmetrical cyanine dye). It should further be appreciated that any of the dyes described herein can also be used in the kits and compositions described herein.

In some embodiments, a dye used in methods provided herein is an intercalating dye. Intercalation refers to the reversible inclusion of a molecule (or group of molecules) between two other molecules (or groups of molecules). Thus, an intercalating dye in the context of the present disclosure refers to dye molecules that become positions between two adjacent nucleotide base pairs of a double-stranded nucleic acid. Dye molecules of an intercalating dye, in some embodiments, interact with the nucleotide base pairs. In some embodiments, intercalating dyes are aromatic dyes. In some embodiments, intercalating aromatic dyes have a planar ring structure and have distinct fluorescence emission spectra. The fluorescence is indicative of the electron delocalization of the intercalating agent and is affected by the inductive effect of substituent groups attached to the dye and by quenching agents. When the aromatic dye is dissolved in an aqueous or aqueous/organic solution, it is believed that the water in the solution significantly quenches the fluorescence of the dissolved aromatic dye by raising the ground-energy-state of the aromatic dye to a level higher than when the dye is in an organic medium. If the aromatic dye intercalates double-stranded nucleic acid, the dye becomes shielded from water. This is because the hybrid contains a relatively hydrophobic interior (the bases) and a hydrophilic exterior (the phosphates). The water thus aggregates at the exterior of the hybrid, and not at the interior. Because the fluorescence emission of the intercalating dye is no longer quenched by the water, the ground-energy-state shifts to a lower energy level, and the result is that the fluorescence emission maximum shifts to a longer wavelength. The fluorescence intensity of the dye upon intercalation is also enhanced many-fold. This shift in fluorescence emission and intensity is thus a property change that is generated in the entity, only upon intercalating a double-stranded nucleic acid.

Any fluorescent aromatic dye that can intercalate double-stranded nucleic acid, and which undergoes a shift in fluorescence emission upon intercalation, can be used in the methods and kits provided herein. Examples of aromatic dyes for use as provided herein include, but are not limited to, phenanthridines, acridines and anthracylines. Examples of phenanthridines include, but are not limited to, ethidium, propidium, butidium, pertidium, dimidium, and phenidium.

Commercially available dyes include SYBR Green I (Corbett Life Science/Qiagen), LC Green (Biofire, Salt Lake City Utah), SYTO9 (Molecular probes, Eugene, Oreg.), Eva Green (Biotium, Hayward, Calif.), Chromofy and Bebo (TATAA Biocenter, Gotenborg, Sweden), and Roche High Resolution Melting Dye (Roche).

It should be appreciated that methods provided herein are not limited to the use of fluorescent dyes. Any dye that can emit a signal upon binding to double-stranded nucleic acid, while not emitting a signal when bound to single stranded nucleic acid and/or not being able to bind to single stranded nucleic acid, can be used in the methods provided herein.

It also should be appreciated that in some embodiments a dye can be used if it has a lower emission intensity when bound to double-stranded nucleic acids (e.g., double-stranded DNA) than unbound (or bound to single-stranded nucleic acids) provided a change in emission intensity (e.g., at one or more wavelengths) can be detected when a double-stranded nucleic acid is denatured (e.g., in response to heating).

### Noncoding Control Region (NCCR) of the JCV

In one aspect, the disclosure provides methods for determining the presence of a JCV or JCV mutant in a sample. In some embodiments, the disclosure provides methods for determining the presence of a mutation in the NCCR (Noncoding Control Region) of a JCV. In some embodiments, the methods provided herein include the element of determining the melting temperature of a double-stranded JCV nucleic acid. In some embodiments, the double-stranded JCV nucleic acid comprises the NCCR (Noncoding Control Region) of the JCV. In some embodiments, the double-stranded JCV nucleic acid analyzed in the methods provided herein, is a part of a NCCR (Noncoding Control Region). In some embodiments, the nucleic acid of the NCCR region of a JCV is amplified by using primers that fall just outside the NCCR region. In some embodiments, the nucleic acid of the NCCR region of a JCV is amplified by using primers that are inside the NCCR region but that still result in the amplification of a signification portion of the NCCR region. In some embodiments, the nucleic acid of the NCCR region of a JCV is amplified by using primers that are inside the NCCR region but that result in the amplification of a portion of the NCCR region that is known to include clinically significant mutations. In some embodiments, the double-stranded JCV nucleic acid of the NCCR is at least 50 nucleotides in length. In some embodiments, the double-stranded JCV nucleic acid of the NCCR is at least 100 nucleotides in length. In some embodiments, the double-stranded JCV nucleic acid of the NCCR is at least 200 nucleotides in length. In some embodiments, the double-stranded JCV nucleic acid of the NCCR is at least 500 nucleotides in length.

The JCV (JCV) is a member of the genus Polyomavirus, which includes JCV, BK virus, WU virus, KI virus, Merkel cell polyomavirus, Simian Virus 40, and mouse polyomavirus. The genome of the JCV is a double-stranded, circular DNA molecule of roughly 5100 bases. The genome encodes 6 proteins and can be divided into 3 segments: early genes, late genes, and a noncoding control region (NCCR; also known as the transcription control region, or TCR). The proteins encoded by the early region genes (small t-antigen and large T-antigen) are involved in viral replication and transcription of the late region genes. The late genes encode the capsid proteins VP1, VP2, and VP3, as well as the regulatory protein agnoprotein. The NCCR includes the origin of replication, as well as sequences that control transcription of both early and late genes.

Within individual hosts, JCV can occur in at least 2 forms: a latent, nonpathogenic form and a virulent neurotropic form. The neurotropic form contains a rearranged NCCR and is typically found in the cerebrospinal fluid (CSF), brain, or blood of PML patients. The nonpathogenic form is most frequently detected in urine, and its NCCR is not rearranged (Yogo et al., J. Virol. 1990, 64: 3139-43). NCCR rearrangements generally involve deletions and duplications of specific sequence elements (reviewed for instance by Yogo and colleagues in Human polyomaviruses: molecular and clinical prospectives, NY, N.Y.: Wiley-Liss, 2001: 127-148), and are thought to play a role in the pathogenesis of the virus by altering its cellular tropism. Thus, determining if mutations are present in the NCCR region provides a method for evaluating if a virulent neurotropic form of the JCV is present in a subject (and take appropriate decisions on therapy if so desired).

### Nucleic Acid Amplification

In one aspect, the disclosure provides methods for determining the presence of a JCV or JCV mutant in a sample. In some embodiments, the methods provided herein include the element of determining the melting temperature of a double-stranded JCV nucleic acid. In some embodiments, the double-stranded JCV nucleic acid is amplified. In some embodiments, amplifying comprises contacting the sample comprising nucleic acid of a JCV with at least two primers that can hybridize to the nucleic acid of a JCV.

In one aspect, the disclosure provides methods for determining the presence of nucleic acids in a sample (mutants) that differ in sequence from a wild-type (non-mutant) sequence. In one aspect, the disclosure provides methods for determining the presence of mutant nucleic acids in a sample. In some embodiments, the mutant nucleic acid is a JCV mutant nucleic acid. In one aspect, the disclosure provides methods for determining the amount of nucleic acid in a sample. In some embodiments, the nucleic acid is DNA. In some embodiments, the nucleic acid is viral nucleic acid. In some embodiments, the nucleic acid is viral DNA. In some embodiments, the nucleic acid is JCV DNA. In some embodiments, the nucleic acid is isolated from a biological sample. In some embodiments, the nucleic acid is isolated from a blood sample or a CSF sample. It should further be appreciated that aspects of the invention (e.g., amplification and or high resolution melting) may be used in combination with any suitable technique for isolating nucleic acid (e.g., from blood or CSF).

In one aspect, the disclosure provides methods for determining the presence of a JCV and JCV mutant in a sample. In some embodiments, the methods provided herein include a step of amplifying the nucleic acid of JCV present in a sample to be interrogated. In some embodiments, the methods provided herein include a step of amplifying the nucleic acid of JC wild type virus present in the sample. In some embodiments, the methods provided herein include a step of amplifying the nucleic acid of JC mutant virus present in the sample. It should further be appreciated that the nucleic acids may be isolated from the sample or partially purified prior to amplification.

In some embodiments, the methods of amplifying the nucleic acid in a sample, or isolated or purified from a sample include a Polymerase Chain Reaction (PCR). In some embodiments, PCR primers allow for amplification of the NCCR region of JCV. In some embodiments, the primers comprise or consist of the following nucleic acid sequences: GGCCTCGGCCTCCTGTAT (SEQ ID NO:1) and CCACTTCCCCTTGTGCTTT (SEQ ID NO:2). In some embodiments, the primers comprise or consist of the following nucleic acid sequences: CGGCCTCGGCCTCCTGTATATA (SEQ ID NO:3) and TCCACTTCCCCTTGTGCTTTGT (SEQ ID NO:4). In some embodiments, the PCR (or other amplification) reaction is performed in the presence of an intercalating dye. However, it should be appreciated that an intercalating dye can be added after an amplification reaction, but prior to a melting analysis as described herein as aspects of the disclosure are not limited in this respect.

### Nucleic Acids

In one aspect, the disclosure provides methods, kits and compositions for determining the presence of JCV mutant in a sample. In some embodiments, the methods, kits and compositions provided herein include the element of amplifying a nucleic acid (e.g., of a JCV or JCV mutant) by using primers. In some embodiments, the primers are nucleic acid primers. In some embodiments, the primers have a sequence comprising SEQ ID NO:1 and SEQ ID NO:2. In some embodiments, the primers have a sequence comprising SEQ ID NO:3 and SEQ ID NO:4.

In some embodiments, the double-stranded JCV nucleic acid used in the methods provided herein is at least 50 nucleotides in length. In some embodiments, the double-stranded JCV nucleic acid is at least 100 nucleotides in length. In some embodiments, the double-stranded JCV nucleic acid is at least 500 nucleotides in length. In some embodiments, the double-stranded JCV nucleic acid is at least 50, at least 100, at least 200, at least 300, at least 400, at least 500, at least 1000, at least 10,000 or more, nucleotides in length.

In one aspect, the disclosure provides isolated nucleic acids. In some embodiments, nucleic acids are useful in the methods for determining the presence of a JCV or a JCV mutant in a sample. In some embodiments, the nucleic acids are complementary to JCV sequences but not to sequences from other viruses. In some embodiments, the nucleic acids useful for methods for determining the presence of a JCV or a JCV mutant in a sample are designed to detect conserved JCV regions (e.g., the nucleic acids are complementary, for example 100% complementary to, a conserved JCV genomic regions). In some embodiments, the nucleic acids are complementary to the noncoding control region (NCCR) of a JCV. In some embodiments, the nucleic acids are complementary to the NCCR of a JCV as described in Reid et al. (Journal of Infectious Diseases, 2011, 2204: 237-244). In some embodiments, the nucleic acids allow for the detection of the presence of JCV mutants of the NCCR region regardless of whether other variant sequences are present in the JCV genome. In some embodiments, the nucleic acids are primers directed to (e.g., complementary to, for example 100% complementary to) regions of the NCCR, or regions that allow for the amplification of the NCCR region. In some embodiments, the nucleic acids allow for the determination of the amount of JCV in a sample by PCR. In some embodiments, the isolated nucleic acid comprises SEQ ID NO:1. In some embodiments, the isolated nucleic acid comprises SEQ ID NO:2. In some embodiments, the isolated nucleic acid consists of SEQ ID NO:1. In some embodiments, the isolated nucleic acid consists of SEQ ID NO:2. In some embodiments, the isolated nucleic acid comprises SEQ ID NO:3. In some embodiments, the isolated nucleic acid comprises SEQ ID NO:4. In some embodiments, the isolated nucleic acid consists of SEQ ID NO:3. In some embodiments, the isolated nucleic acid consists of SEQ ID NO:4. It should be appreciated that the primers do not need to be 100% complementary, and, in some embodiments, primers that hybridize under high stringency conditions can be used as well. Guidance for performing hybridization reactions can be found in Current Protocols in Molecular Biology, John Wiley & Sons, N.Y. (1989), 6.3.1-6.3.6, which is incorporated by reference. High stringency hybridization conditions include hybridization in 6×SSC at about 45° C., followed by one or more washes in 0.2×SSC, 0.1% SDS at 65° C., or substantially similar conditions.

In some embodiments, the isolated nucleic acid is a nucleic acid primer comprising SEQ ID NO:1. In some embodiments, the isolated nucleic acid is a nucleic acid primer comprising SEQ ID NO:2. In some embodiments, the isolated nucleic acid is a nucleic acid primer comprising SEQ ID NO:3. In some embodiments, the isolated nucleic acid is a nucleic acid primer comprising SEQ ID NO:4. In some embodiments, the isolated nucleic acid is a nucleic acid primer that consists of SEQ ID NO:1. In some embodiments, the isolated nucleic acid is a nucleic acid primer that consists of SEQ ID NO:2. In some embodiments, the isolated nucleic acid is a nucleic acid primer that consists of SEQ ID NO:3. In some embodiments, the isolated nucleic acid is a nucleic acid primer that consists of SEQ ID NO:4. The isolated nucleic acids disclosed herein may further have one or more functionalities (e.g., a fluorescent label or other detectable label). In some embodiments, the nucleic acid primer is GGCCTCGGCCTCCTGTAT (SEQ ID NO:1). In some embodiments, the nucleic acid primer is CCACTTCCCCTTGTGCTTT (SEQ ID NO:2). In some embodiments, the nucleic acid primer is CGGCCTCGGCCTCCTGTATATA (SEQ ID NO:3). In some embodiments, the nucleic acid primer is TCCACTTCCCCTTGTGCTTTGT (SEQ ID NO:4). It should further be appreciated that any of the nucleic acids provided herein can also be an element of the kits described herein. For example, in some embodiments, nucleic acids of the present disclosure are provided in an aqueous buffer. In some embodiments, nucleic of the present disclosure are lyophilized.

### Samples

In one aspect, the disclosure provides methods, kits and compositions for determining the presence of JCV mutant in a sample. In some embodiments, the sample is a biological sample. In some embodiments, the sample is from a subject suspected of being infected with JCV. In some embodiments, the sample is from a subject suspected of being infected with a JCV mutant. In some embodiments, the sample is a blood sample. In some embodiments, the sample is a Cerebrospinal Fluid (CSF) sample. In some embodiments, the sample is a urine sample.

In one aspect, the methods provided herein allow for determining the presence of a JCV or JCV mutant in a sample. In one aspect, the methods provided herein allow for determining the presence of a JCV or JCV mutant in a biological sample.

The methods provided herein allow for the determining the presence of JCV and JCV mutants in biological samples. It is thought that JCV persists mostly in the kidneys and urine in the absence of PML, and that PML is associated with the presence of JCV in the brain. The methods and compositions of the invention are also useful to determine the presence of JCV or JCV mutants in urine, blood, renal tissue and Cerebrospinal Fluid (CSF) sample, or other patient samples.

Methods for determining the presence of JCV and JCV mutants in biological samples may be carried out on any suitable biological sample. In some embodiments, a sample may be obtained from a subject and directly processed and assayed as described herein. In some embodiments, cells may be isolated from a biological sample and grown in culture prior to analysis. As used herein, a subject may be a human or a non-human animal, including, but not limited to a non-human primate, cow, horse, pig, sheep, goat, dog, cat, or rodent. In some embodiments, the subject is a human. Methods of the invention may be used to determine the presence of JCV and JCV mutants in subjects not yet diagnosed with PML. Methods of the invention may be used to determine the presence of JCV and JCV mutants in subjects not yet diagnosed as being infected with JCV. In addition, methods of the invention may be applied to subjects who have been diagnosed with PML and/or infection by a JCV mutant. A sample may comprise one or more cells. A sample may originate directly from a subject or from a cell culture. A sample may be processed (e.g., to prepare a cell lysate, or plasma concentrate) or partially processed prior to use in methods of the invention. In some embodiments, a sample from a subject or culture may be processed to obtain nucleic acids to determine the presence of JCV and JCV mutants. Thus, an initial step in an assay may include isolation of a nucleic acid from a cell, tissue, and/or other sample. Extraction of nucleic acids may be by any suitable means, including routine methods used by those of ordinary skill in the art such as methods that include the use of detergent lysates, sonification, and/or vortexing with glass beads, etc.

As used herein, a “sample” means any animal material containing DNA or RNA or protein, such as, for example, tissue or fluid isolated from an individual (including without limitation plasma, serum, cerebrospinal fluid, urine, lymph, tears, saliva and tissue sections) or from in vitro cell culture constituents. A sample containing nucleic acids may contain of deoxyribonucleic acids (DNA), ribonucleic acids (RNA), or copolymers of deoxyribonucleic acids and ribonucleic acids or combinations thereof. A sample containing polypeptides may contain peptides and/or proteins. A sample may have been subject to purification (e.g., extraction) and/or other treatment.

Methods for isolating nucleic acids from a biological sample such as a blood sample and CSF sample are known in the art. Methods for isolating nucleic acids from a biological sample can include the use of one or more components from commercially available nucleic acid isolation kits, such as kits provided by Qiagen, Promega and Epicentre. In some embodiments, the methods provided herein use one or more components from the QIAamp MinElute Virus Spin Kit (Cat #57704, Qiagen). However, it should be appreciated that the methods disclosed herein can also be practiced with components from other commercially available nucleic acid kits.

As used herein, the term “biological sample” may refer to tissue, cells or component parts (e.g., body fluids, including but not limited to blood, mucus, lymphatic fluid, synovial fluid, cerebrospinal fluid, saliva, amniotic fluid, amniotic cord blood, urine, stool, vaginal fluid, and semen, etc.) of a subject. A “biological sample” may also refer to a homogenate, lysate, or extract prepared from tissues, cells or component parts, or a fraction or portion thereof, including, but not limited to, for example, plasma, serum, spinal fluid, lymph fluid, urine, the external sections of the skin, respiratory, intestinal, and genitourinary tracts, tears, saliva, stool, milk, blood cells, tumors, organs, or CNS biopsies.

Sample sources may include, without limitation, tissues, including, but not limited to lymph tissues; body fluids (e.g., blood, lymph fluid, etc.), cultured cells; cell lines; histological slides; and tissue embedded in paraffin. The term “tissue” as used herein includes both localized and disseminated cell populations including, but not limited to: brain, heart, serum, breast, colon, bladder, epidermis, skin, uterus, prostate, stomach, testis, ovary, pancreas, pituitary gland, adrenal gland, thyroid gland, salivary gland, mammary gland, kidney, liver, intestine, spleen, thymus, bone marrow, trachea, and lung. Biological fluids include, but are not limited to, blood, lymph fluid, cerebrospinal fluid, tears, saliva, urine, and feces. Invasive and non-invasive techniques can be used to obtain such samples and are well documented in the art. A control sample may include a bodily fluid, a cell, a tissue, or a lysate thereof. In some embodiments, a control sample may be a sample from a cell or subject that is free of PML and/or infection by, or exposure to, a JCV mutant. In some embodiments, a control sample may be a sample that is from a cell or subject that has PML and/or has been exposed to a JCV mutant. In some embodiments a control sample is a sample comprising wild-type JCV.

In some embodiments, the biological sample is a cerebrospinal fluid sample (CSF). Cerebrospinal fluid is a fluid that surrounds and protects the brain and the spinal cord. The fluid generally is clear liquid that contains proteins and white blood cells. In some embodiments, CSF is obtained from a subject through a lumbar puncture (spinal tap). A lumbar puncture is a procedure that is unpleasant to a subject and the number of lumbar punctures should be minimized. A variety of disorders that affect the brain and/or the central nervous system, including meningitis, tumors of the brain, and hemorrhaging of the brain, can be diagnosed by analyzing the CSF. Viral infections of the brain, such as infections by a JCV, can be diagnosed by detecting the presence of, and/or quantifying the amount of, viral DNA in the CSF. Because the amount of viral DNA (or viral RNA) in the CSF can be low, it is important to have diagnostic techniques that can accurately detect even small amounts of the virus.

Immuno-Compromised Subjects or Subjects Undergoing Treatment with Immuno-Suppressants

In one aspect, the methods disclosed herein may be particularly useful for determining the presence of a JCV or JCV mutant in a sample obtained from an immuno-compromised subject and/or a subject being treated with one or more immuno-suppressants. Subjects may receive treatment with one or more immunosuppressive agents (also called immuno-suppressants) directed to different diseases or conditions, including one or more of the following non-limiting examples: cancer, organ or tissue transplant, inflammatory conditions or diseases, multiple sclerosis (MS), arthritis, etc., or any combination thereof. Subjects may also be immuno-compromised. Non-limiting examples of immuno-compromised subjects are subjects that are HIV positive or have AIDS or lymphoma or any other condition resulting in a suppression of the immune response.

The term “immuno-suppressive agent” as used herein refers to substances that act to suppress or mask the immune system of a subject being treated herein. Immuno-suppressive agents may be substances that suppress cytokine production, down-regulate or suppress self-antigen expression, or mask the MHC antigens. Examples of such agents include 2-amino-6-aryl-5-substituted pyrimidines (see U.S. Pat. No. 4,665,077); nonsteroidal anti-inflammatory drugs (NSAIDs); ganciclovir, tacrolimus, glucocorticoids such as cortisol or aldosterone, anti-inflammatory agents such as a cyclooxygenase inhibitor, a 5-lipoxygenase inhibitor, or a leukotriene receptor antagonist; purine antagonists such as azathioprine or mycophenolate mofetil (MMF); alkylating agents such as cyclophosphamide; bromocryptine; danazol; dapsone; glutaraldehyde (which masks the MHC antigens, as described in U.S. Pat. No. 4,120,649); anti-idiotypic antibodies for MHC antigens and MHC fragments; cyclosporin A; steroids such as corticosteroids or glucocorticosteroids or glucocorticoid analogs, e.g., prednisone, methylprednisolone, and dexamethasone; dihydrofolate reductase inhibitors such as methotrexate (oral or subcutaneous); hydroxycloroquine; sulfasalazine; leflunomide; cytokine or cytokine receptor antagonists including anti-interferon-alpha, -beta, or -gamma antibodies, anti-tumor necrosis factor-alpha antibodies (infliximab or adalimumab), anti-TNF-alpha immunoahesin (etanercept), anti-tumor necrosis factor-beta antibodies, anti-interleukin-2 antibodies and anti-IL-2 receptor antibodies; anti-LFA-1 antibodies, including anti-CD11a and anti-CD18 antibodies; anti-CD20 antibodies (e.g., rituximab, for example available under the trademark RITUXAN); anti-L3T4 antibodies; anti-VLA-4 antibodies (e.g., natalizumab, for example available under the trademark TYSABRI); heterologous anti-lymphocyte globulin; pan-T antibodies, for example anti-CD3 or anti-CD4/CD4a antibodies; soluble peptide containing a LFA-3 binding domain (WO 90/08187 published Jul. 26, 1990); streptokinase; TGF-beta; streptodornase; RNA or DNA from the host; FK506; RS-61443; deoxyspergualin; rapamycin; T-cell receptor (Cohen et al., U.S. Pat. No. 5,114,721); T-cell receptor fragments (Offner et al., Science, 251: 430432 (1991), WO 90/11294, Janeway, Nature, 341: 482 (1989), and WO 91/01133); and T cell receptor antibodies (EP 340,109) such as T10B9. Subjects receiving other immunosuppressive agents may be selected for the methods provided herein as the invention is not limited in this respect.

### Treatment Relating to Immunosuppressants

Methods of selecting treatment may be useful for persons undergoing treatment not directed to PML or JCV infection, but directed to a different condition. In some embodiments, the treatment is a treatment comprising immunosuppressants. In some embodiments, a person suspected of being at risk for developing PML is a person undergoing treatment with immunosuppressants.

In one aspect, the disclosure provides methods for identifying subjects at risk of progressive multifocal leukoencephalopathy (PML). In some embodiments of the methods provided herein, wherein the sample is from a subject, and wherein if the sample is identified as comprising a JCV mutant, the subject is identified as being at risk for developing progressive multifocal leukoencephalopathy (PML).

In one aspect, the disclosure provides methods for identifying subjects that are inappropriate for treatment comprising immunosuppressants. In some embodiments of the methods provided herein, wherein the sample is from a subject, and wherein if the sample is identified as comprising a JCV mutant, the subject is identified as being inappropriate for treatment comprising immunosuppressants. In some embodiments, treatment comprising immunosuppressants comprises treatment including natalizumab.

In one aspect, the disclosure provides methods for identifying subjects that are receiving treatment comprising immunosuppressants as requiring adjustment or termination of treatment comprising immunosuppressants. In some embodiments of the methods provided herein, wherein the sample is from a subject, and wherein if the sample is identified as comprising a JCV mutant, and the subject is receiving treatment comprising immunosuppressants, the subject is identified as requiring adjustment or termination of treatment comprising immunosuppressants. In some embodiments, treatment comprising immunosuppressants comprises treatment including natalizumab.

In some embodiments, determining the presence of a JCV or JCV mutant in a sample obtained from a subject who has received or is receiving treatment not directed to PML, may indicate that the treatment regimen should be adjusted. In some embodiments, determining the presence of a JCV mutant in a sample obtained from a subject who has received or is receiving treatment with immunosuppressants may indicate that the treatment regimen should be adjusted. In some embodiments, determining the presence of a JCV mutant in a sample obtained from a subject who has received or is receiving treatment with immunosuppressants may indicate that the treatment regimen should be terminated or interrupted. In some embodiments, the immunosuppressant is natalizumab. In some embodiments, determining an increase in the amount of JCV mutant in a sample obtained from a subject who has received or is receiving treatment with immunosuppressants in a subject who has received or is receiving treatment with immunosuppressants may indicate that the treatment regimen should be terminated or interrupted. In some embodiments, determining an increase in the number of different JCV mutants in a sample obtained from a subject who has received or is receiving treatment with immunosuppressants in a subject who has received or is receiving treatment with immunosuppressants may indicate that the treatment regimen should be terminated or interrupted.

### Kits

In one aspect, the disclosure provides kits for determining the presence of a JCV or JCV mutant in a sample. In some embodiments, the kit comprises an intercalating dye (e.g., intercalating fluorescent dye) and double-stranded JCV nucleic acid of a non-mutant, or wild-type, JCV (e.g., consisting of or comprising an archetype JCV NCCR region or a trimmed region as described herein).

In some embodiment, a kit comprises a first nucleic acid primer having a sequence comprising SEQ ID NO:1 and a second nucleic acid primer having a sequence comprising SEQ ID NO:2. In some embodiment, the kit comprises a first nucleic acid primer having a sequence comprising SEQ ID NO:3 and a second nucleic acid primer having a sequence comprising SEQ ID NO:4. In some embodiments, the kit comprises a first nucleic acid primer having a sequence comprising SEQ ID NO: 1 and a second nucleic acid primer comprising SEQ ID NO: 4. In some embodiments, the kit comprises a first nucleic acid primer having a sequence comprising SEQ ID NO: 2 and a second nucleic acid primer having a sequence comprising SEQ ID NO: 3.

In some embodiments, one or more primers are labeled (e.g., detectably labeled).

In some embodiments, the kit comprises a reagent comprising a first nucleic acid primer having a sequence comprising SEQ ID NO:1, a second nucleic acid primer having a sequence comprising SEQ ID NO:2 and an intercalating dye. In some embodiments, the kit comprises a reagent comprising a first nucleic acid primer having a sequence comprising SEQ ID NO:3, a second nucleic acid primer having a sequence comprising SEQ ID NO:4 and an intercalating dye. In some embodiments, the kit comprises a reagent comprising a first nucleic acid primer having a sequence comprising SEQ ID NO:1, a second nucleic acid primer having a sequence comprising SEQ ID NO:4 and an intercalating dye. In some embodiments, the kit comprises a reagent comprising a first nucleic acid primer having a sequence comprising SEQ ID NO:2, a second nucleic acid primer having a sequence comprising SEQ ID NO:3 and an intercalating dye.

In some embodiments, the kit comprises one or more reagents each comprising a first nucleic acid primer having a sequence comprising SEQ ID NO:1, a second nucleic acid primer having a sequence comprising SEQ ID NO:2, an intercalating dye, a double-stranded JCV nucleic acid of a non-mutant (e.g., an archetype) JCV, or any combination of two or more thereof. In some embodiments, the kit comprises one or more reagents each comprising a first nucleic acid primer having a sequence comprising SEQ ID NO:3, a second nucleic acid primer having a sequence comprising SEQ ID NO:4, an intercalating dye, double-stranded JCV nucleic acid of a non-mutant (e.g., an archetype) JCV, or any combination of two or more thereof. In some embodiments, the kit comprises one or more reagents each comprising a first nucleic acid primer having a sequence comprising SEQ ID NO:1, a second nucleic acid primer having a sequence comprising SEQ ID NO:4, an intercalating dye, a double-stranded JCV nucleic acid of a non-mutant (e.g., an archetype) JCV, or any combination of two or more thereof. In some embodiments, the kit comprises one or more reagents each comprising a first nucleic acid primer having a sequence comprising SEQ ID NO:2, a second nucleic acid primer having a sequence comprising SEQ ID NO:3, an intercalating dye, a double-stranded JCV nucleic acid of a non-mutant (e.g., an archetype) JCV, or any combination of two or more thereof.

In some embodiments, the kit comprises one or more reagents each comprising a first nucleic acid primer having a sequence comprising SEQ ID NO:1, a second nucleic acid primer having a sequence comprising SEQ ID NO:2, an intercalating dye, a double-stranded JCV nucleic acid of a non-mutant (e.g., an archetype) JCV, nucleic acid amplification (e.g., PCR) reaction components, or any combination of two or more thereof. In some embodiments, the kit comprises one or more reagents each comprising a first nucleic acid primer having a sequence comprising SEQ ID NO:3, a second nucleic acid primer having a sequence comprising SEQ ID NO:4, an intercalating dye, double-stranded JCV nucleic acid of a non-mutant (e.g., an archetype) JCV, nucleic acid amplification reaction components, or any combination of two or more thereof. In some embodiments, the kit comprises one or more reagents each comprising a first nucleic acid primer having a sequence comprising SEQ ID NO:1, a second nucleic acid primer having a sequence comprising SEQ ID NO:4, an intercalating dye, a double-stranded JCV nucleic acid of a non-mutant (e.g., an archetype) JCV, nucleic acid amplification reaction components, or any combination of two or more thereof. In some embodiments, the kit comprises one or more reagents each comprising a first nucleic acid primer having a sequence comprising SEQ ID NO:2, a second nucleic acid primer having a sequence comprising SEQ ID NO:3, an intercalating dye, a double-stranded JCV nucleic acid of a non-mutant (e.g., an archetype) JCV, nucleic acid amplification reaction components, or any combination of two or more thereof.

It should be appreciated that in some embodiments, a reagent may include one, two, three, four, or more of the different components in a pre-mixed preparation. In other embodiments, different components are provided as separate reagents in a kit (e.g., along with instructions for mixing them for use in a method described herein).

Nucleic acid amplification reaction components may include, for example, buffers, nucleotides (e.g., dNTPs), enzymes and/or salt for polymerase chain reaction or other amplification reaction.

In some embodiments, one or more primers may exist in the form of an aqueous solution wherein the concentration of each primer may be 0.1 nM to 1.0 mM. In some embodiments, the concentration of an aqueous primer may be 0.1 μM to 1 μM, or 0.1 μM to 0.5 μM. It should also be appreciated that, in some embodiments, one or more primers may exist in a dry, lyophilized state wherein the amount of primer may range from 0.04 pmol to 500 nmol. In some embodiments, the intercalating dye may exist in the form of an aqueous solution wherein the concentration of dye may range from 0.1 nM to 200 mM. In some embodiments, the intercalating dye may exist in a dry, lyophilized form wherein the mass of the intercalating dye may range from 1 pg to 1 mg. In some embodiments, the double-stranded JCV nucleic acid of a non-mutant, or wild-type, JCV may exist in the form of an aqueous solution, wherein the concentration of the nucleic acid may range from 0.5 pg/μL to 1.0 μg/μL. In some embodiments, the double-stranded JCV nucleic acid of a non-mutant, or wild-type, JCV may exist in a dry, lyophilized form wherein the mass of the nucleic acid may range between 0.5 pg to 1.0 μg.

In some embodiments, each primer or primer pair is provided in a separate, individual container. In some embodiments, each primer or primer pair is provided in a mixture with other reagents of a kit (e.g., one or more nucleic acid amplification reagents (e.g., buffers, nucleotides, enzymes and/or salt).

Reagents for use in nucleic acid amplification reactions (e.g., PCR) and HRM assays may also be included in kits. In some embodiments, the kit may include instructions for determining the presence of a JCV or JCV mutant in a sample. The kit may also include control values (e.g., reference numbers) that can be used for interpreting results of methods used in the invention.

In some embodiments, the kits contain one or more components for isolating and preparing nucleic acids and/or one or more components for assaying for determining the presence and/or amount of a JCV mutant. In some embodiments, a kit contains one or more buffers and/or other solutions for isolating JCV particles and/or JCV nucleic acid from a biological sample (e.g., a blood sample or a CSF sample), and optionally instructions for performing one or more isolation steps. In some embodiments, a kit contains one or more reagents for determining the presence of a JCV nucleic acid in a sample (e.g., a mutant JCV). For example, a kit may include nucleic acid having a specified sequence. In some embodiments, the nucleic acid (e.g., a nucleic acid primer) may be provided as a dried powder (e.g., a lyophilized preparation). In some embodiments, the nucleic acid may be provided in solution. The solution may be diluent, a buffer, a salt solution, an aqueous solution, or other solution, including, for example, water. The solution may contain a known (e.g., predetermined) concentration of the nucleic acids. The kit may contain instructions for diluting the nucleic acid solution to one or more appropriate concentrations defined for one or more specified ingredients that are to be marked for subsequent authentication or quality control purposes. In some embodiments, a kit may contain one or more oligonucleotides (e.g., PCR primers) that can be used to detect the presence, in a biological sample (e.g., a blood sample or a CSF sample), of a nucleic acid having a specified sequence. In some embodiment, the kit includes primers have a sequence comprising SEQ ID NO:1 and SEQ ID NO:2. In some embodiment, the kit includes primers have a sequence comprising SEQ ID NO:3 and SEQ ID NO:4.

A kit also may contain one or more enzymes and/or other reagents for performing nucleic acid isolation, detection, and/or quantification assay disclosed herein. In some embodiments, a kit may contain a reference nucleic acid having a specified sequence of interest (e.g., a non-mutant JCV). A reference level (e.g., information about a reference level) and/or a reference sample containing a nucleic acid at a reference level also may be provided in a kit. Such information and/or nucleic acids can be used as controls. In some embodiments, a kit also may include instructions for isolating nucleic acids (e.g., JCV nucleic acids) from a patient sample (e.g., a blood sample or a CSF sample).

In some embodiments, a kit comprises at least one container means having disposed therein one or more reagents (e.g., wash buffers, lysis buffers, proteases, elution buffers, etc.) and/or nucleic acids described herein (e.g., PCR primers, intercalating dyes, non-mutant JCV nucleic acid, etc.). In certain embodiments, the kit further comprises other containers comprising one or more other reagents or probes. A kit also may contain detection reagents. In some embodiments, the kit contains an intercalating agent.

In one aspect, a compartmentalized kit includes any kit in which reagents are contained in separate containers. Such containers include small glass containers, plastic containers or strips of plastic or paper. Such containers allow the efficient transfer of reagents from one compartment to another compartment such that the samples and reagents are not cross-contaminated and the agents or solutions of each container can be added in a quantitative fashion from one compartment to another. In some embodiments, a kit may include a container which will accept the test sample, a container which contains the probe or primers used in the assay, containers which contain wash reagents (such as phosphate buffered saline, Tris-buffers, and the like), and containers which contain the reagents used to detect the hybridized probe, amplified product, or the like.

In some embodiments, a kit contains reagents in a multi-well plate.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference, in particular for the teaching that is referenced hereinabove.

### EXAMPLES

### Materials and Methods

### PCR for High Resolution Melt Curve Analysis for Determination of JCV NCCR Mutations

### Instrument Used:

Roche LightCycler 480 (type I)
Detection Format: SYBR Green I/HRM Dye (483-533)—Dynamic integration time

### PCR Reaction Conditions:

96-well plate (Cat #04 729 692 001, Roche)
50 μL total reaction volume

### Primer Sequence:

NCCR1_Forward Primer: GGCCTCGGCCTCCTGTAT (SEQ ID NO: 1)
NCCR1_Reverse Primer: CCACTTCCCCTTGTGCTTT (SEQ ID NO: 2)

| OLIGO | start | length | Tm | GC% | any | 3′ | sequence |
|---|---|---|---|---|---|---|---|
| Forward primer | 100 | 18 | 62.50 | 66.67 | 6.00 | 2.00 | GGCCTCGGCCTCCTGTAT |
| Reverse primer | 315 | 19 | 60.10 | 52.63 | 3.00 | 0.00 | CCACTTCCCCTTGTGCTTT |

### Product Size: 216 bp

Primers were obtained by custom order from Integrated DNA Technologies

### Reaction Components:

| | | | | Final | |
|---|---|---|---|---|---|
| Component | Stock Conc. | Volume added | | conc. | |
| Roche LC480 | 2X | 25 | μL | 1X | |
| HRM Master Mix | | | | | |
| MgCl₂ | 25 mM | 6 | μL | 3.0 | mM |
| NCCR1_FP | 20 μM | 0.75 | μL | 300 | nM |
| NCCR1_RP | 20 μM | 0.75 | μL | 300 | nM |
| DNase/RNase free H₂O | | 7.5 | μL | | |
| DNA Template | | 10 | μL | | |
| | | 50 | μL total | | |

Master Mix, MgCl₂, and H₂O come as part of Cat #04 909 631 001, Roche.

### PCR Conditions:

| Program | Target (° C.) | Acquisition Mode | Hold (hh:mm:ss) | Ramp Rate (° C./s) | Acquisitions (per ° C.) | Cycles |
|---|---|---|---|---|---|---|
| Pre-incubation | 95 | None | 0:10:00 | 4.4 | — | — |
| Amplification | 95 | None | 0:00:10 | 4.4 | — | 50 |
| | 60 | None | 0:00:15 | 2.2 | — | |
| | 72 | Single | 0:00:09 | 4.4 | — | |
| Melting | 95 | None | 0:01:00 | 4.4 | — | — |
| | 40 | None | 0:01:00 | 2.2 | — | |
| | 65 | None | 0:00:01 | 1 | — | |
| | 95 | Continuous | — | 0.02 | 25 | |
| Cooldown | 40 | None | 0:00:10 | 2.2 | — | — |

Software Analysis: Roche LightCycler 480 Software v1.5 with Gene Scanning Add-on

### Analysis 1: Abs Quant/2^{nd} Derivative Max

### Analysis 2: Gene Scanning

Melting curve normalization ranges: 76-78° C. for pre-melt, 90-91° C. for post-melt
Temperature shift threshold set to 0

### Use Difference Plot or Melting Peaks

### Example 1

### Differentiation of Mad-1 and Archetype NCCRs

Twelve plasmids, each representing one of 12 patient-derived NCCR (Noncoding Control Region) sequences cloned into pCR4-TOPO vectors and transformed into TOP10 cells using the TOPO TA Cloning Kit for sequencing (Cat. 45-0030, Invitrogen), were obtained in plasmid form and retransformed into TOP10 cells using a standard heat-shock method (See Reid et al, J Infect Dis. 2011 Jul. 15; 204(2):237-44 for more information regarding the original cloning).

The plasmids were purified from the retransformed cultures using the Qiagen Spin Miniprep Kit (Cat. 27104, Qiagen) according to protocol, which usually yielded about 150 ng/μL. The plasmids are denoted in these experiments by the names 144-01, 146-01 (Mad-1), 149-13 (archetype), 161-03, 168-16, 173-01, 224-01, 225-01, 229-01, 229-01, 229-24, 234-24, and 242-01. All of these plasmids have rearranged NCCR genotypes except for 149-13 (archetype).

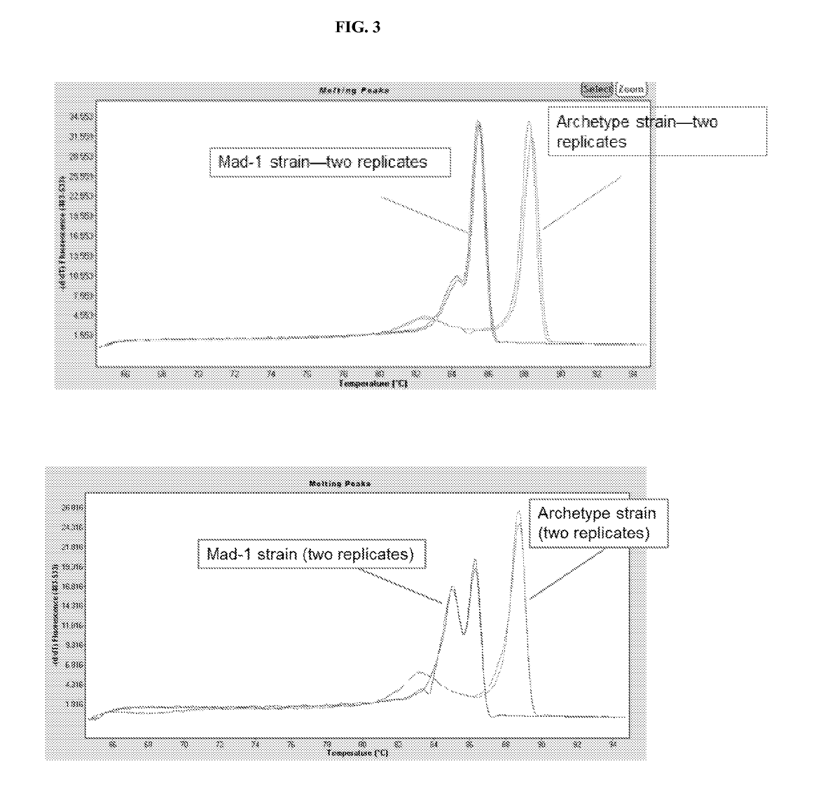

Stocks meant for PCR reactions were generated for each plasmid by a 1:5000 dilution of the miniprep eluate. The curves shown in FIG. 3 are from plasmid numbers 146-01 (Mad-1 strain) and 149-13 (archetype strain). Two replicates of each genotype are shown. Amounts of plasmids in each reaction were determined to be 4.33×10⁷ copies/reaction for 146-01 (Mad-1) and 2.78×10⁶ copies/reaction for 149-13 (archetype).

### Example 2

### Differentiation of a Range of NCCR Genotypes

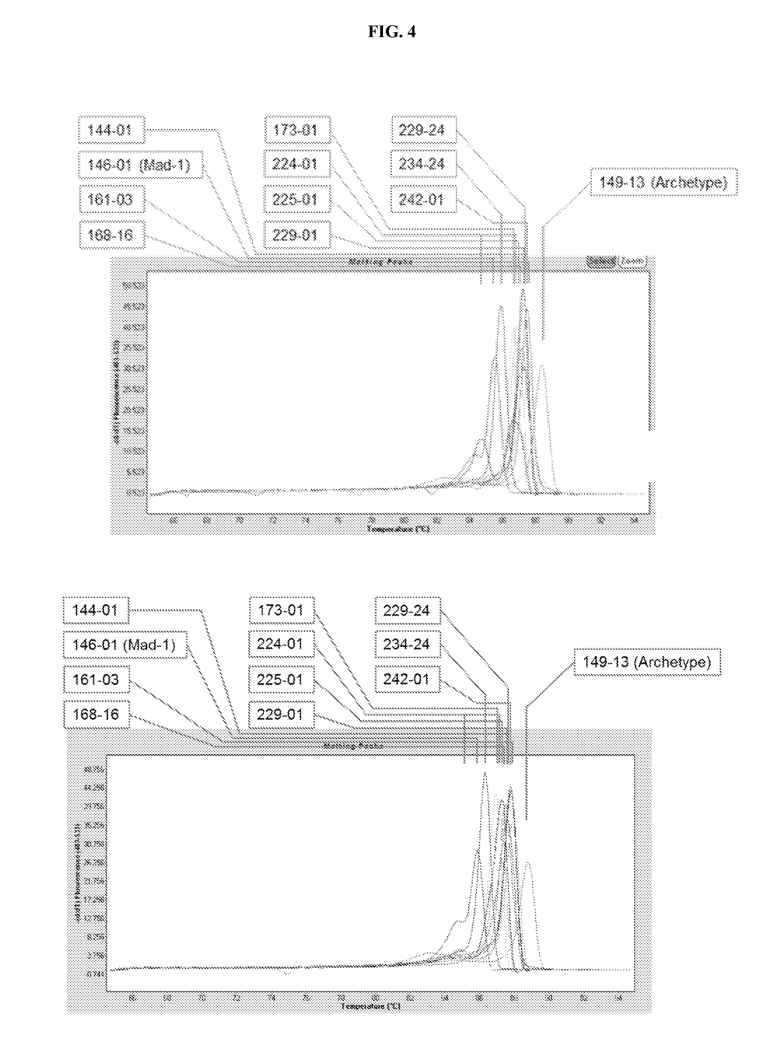

Similarly as discussed in Example 1 above, all 12 plasmid stocks were run using the HRM method provided herein. The results are depicted in FIG. 4. One replicate of each genotype is shown. Amounts of plasmids in each reaction were estimated to be on the order of 10⁷ copies/reaction. Two of the plasmids (229-01 and 229-24) are from different clones of virus from the same patient, and have the same NCCR sequence—these curves overlap, while the others are more unique.

### Example 3

### Genotyping Mixtures of Different NCCR Genotypes

Mixtures of 146-01 (Mad-1) and 149-13 (archetype) were generated according to Table 1 and HRM experiments were performed.

TABLE 1
Mixtures of Mad-1 and archetype

| | | | Amt. of 146-01 | | Amt. of 149-13 |
|---|---|---|---|---|---|
| | | Vol. of 146-01 | (Mad-1) | Vol. of 149-13 | (arche) |
| Mix name | Curve\|certifier | (Mad-1) (μL) | (copies/reaction) | (arche) (μL) | (copies/reaction) |
| Pure Mad-1 | A | 10 | 866000 | 0 | 0 |
| 1 | B | 8 | 692800 | 2 | 11120 |
| 2 | C | 7 | 606200 | 3 | 16680 |
| 3 | D | 6 | 519600 | 4 | 22240 |
| 4 | E | 5 | 433000 | 5 | 27900 |
| 5 | F | 4 | 346420 | 6 | 33380 |
| 6 | G | 3 | 259800 | 7 | 38920 |
| 7 | H | 2 | 273200 | 8 | 44480 |
| 8 | I | 1 | 86600 | 9 | 50040 |
| Pure archetype | J | 0 | 0 | 10 | 55600 |

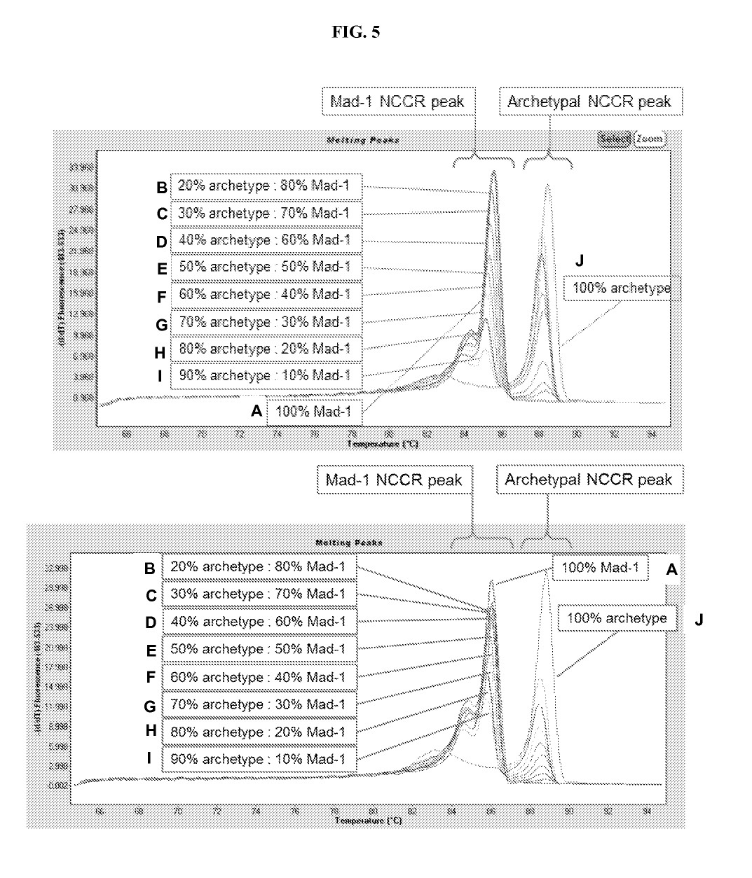

The results are depicted in FIG. 5. The percentages denoted in FIG. 5 are volumetric percentages; the actual copy numbers are given in Table 1.

### Example 4

### Genotyping Across a Dilution Series for Four Different Genotypes

A serial dilution series was generated for each of the genotypes labeled 146-01 (Mad-1), 149-13 (archetype), 173-01, and 224-01, starting with a stock defined as a 1:5000 dilution of the eluate of a Qiagen Spin Miniprep Kit (Cat. 27104, Qiagen). The stocks were determined (according to extrapolation from a standard curve of JC Virus (MAD1 Strain) Quantitated Viral DNA (Cat. 08-943-250, Advanced Biotechnologies)) to consist of the values shown in column 2 in Table 2. Each dilution series was generated via serial 10-fold dilutions (5 μL of previous dilution into 45 μL H2O), and consisted of 1:10, 1:100, 1:1000, 1:10⁴, 1:10⁵, 1:10⁶, and 1:10⁷ dilutions of that genotype's stock. Thus, with 10 μL of each sample used as template in its PCR reaction, each dilution series ranged from the values in column 3 to the values in column 4 below:

TABLE 2
Serial dilutions

| | Stock conc. | Series range max. | Series range min. |
|---|---|---|---|
| Plasmid | (copies/mL) | (copies/rxn) | (copies/rxn) |
| 146-01 (Mad-1) | 4.33E+09 | 4.33E+07 | 4.33 |
| 149-13 (arche.) | 2.78E+08 | 2.78E+06 | 0.278 |
| 173-01 | 2.07E+09 | 2.07E+07 | 2.07 |
| 224-01 | 1.01E+09 | 1.01E+07 | 1.01 |

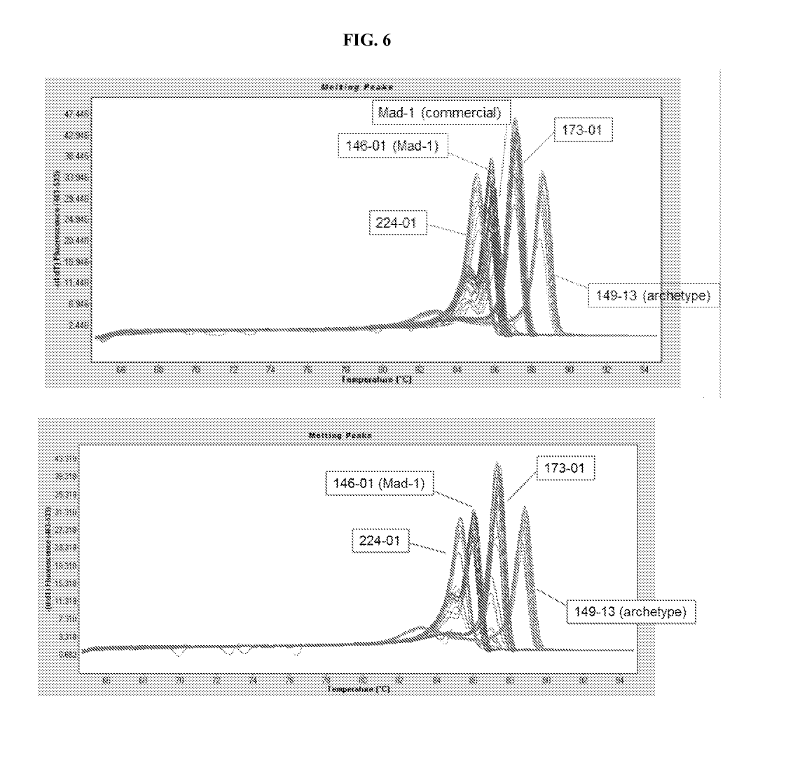

The 1:10⁷ dilutions amplified in 2/2 replicates for 146-01 (Mad-1), in 1/2 replicates for 173-13 (archetype), in 1/2 replicates for 173-01, and in 2/2 replicates for 224-01; the 1:10⁶ dilutions amplified in 1/2 replicates for 173-13 (archetype) and 2/2 replicates for the other genotypes; and all other reactions amplified in both replicates. The results are depicted in FIG. 6. Negative reactions are not shown. Reactions were quantitated using the following standard curve (copies/mL of JCV (MAD1 Strain) Quantitated Viral DNA (Cat. 08-943-250, Advanced Biotechnologies)). See also Table 3 below.

TABLE 3
standard dilution curve

| Standard | Conc. |
|---|---|
| name | (copies/mL) |
| Stock | 130000000 |
| P1 | 100000000 |
| P2 | 10000000 |
| P3 | 1000000 |
| 1 | 100000 |
| 2 | 10000 |
| 3 | 1000 |
| 5 | 100 |
| 8 | 10 |

### Example 5

### Demonstration of Different HRM Signatures for Different NCCR Rearrangements in Clinical Samples

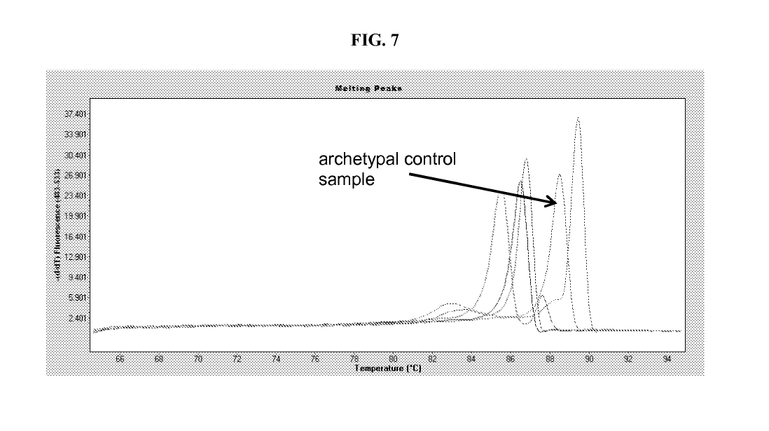

CSF samples were obtained from late-stage PML patients. One mL of each sample was extracted using a MinElute Virus Kit (Qiagen) with slight protocol modifications. Extraction eluates were originally loaded directly into the PCR-HRM reaction; however, because the samples were run again using the new primers, there was very little eluate remaining. Each eluate tube, therefore, had 21 μL water added to it, was mixed, and then was used as if this new addition were the eluate. Positives were of course amplified non-quantitatively, but the HRM signature was unaffected. Archetype control in FIG. 7 is in green; all other curves are clinical samples. The samples also shown by sequencing to have rearranged NCCRs.

### Example 6

### Specificity to JC Virus

The listed viruses were obtained either as cloned viral DNA or, in the case of retroviruses, as infected cell DNA from Advanced Biotechnologies Inc. Samples were diluted to 5000 copies/mL if they came quantitated and to 0.78 ng/μL if they came as infected cell DNA. No amplification was observed for the non-JCV viruses, as shown in Table 4, indicating that the assay is specific to JCV. Also, JCV amplification was not significantly inhibited in the presence of these viruses.

TABLE 4

| | | Copies/mL |
|---|---|---|
| Viral DNA | JCV Copies/mL | (+5000 copies/mL |
| (5000 copies/mL) | (No JCV DNA) | JCV DNA) |
| VZV | 0 | 3705 |
| HHV6A | 0 | 3445 |
| HHV7 | 0 | 3860 |
| HIV1 | 0 | 2560 |
| HTLV-I | 0 | 1746 |
| HTLV-II | 0 | 4765 |
| BKV | 0 | 4875 |
| JCV | 4635 | 6310 |

### Example 7

### Comparative Sensitivity and Specificity of Two Different Primer Sets

### PCR Reaction Conditions:

96-well plate (Cat #04 729 692 001, Roche)
30 μL total reaction volume

### Primer Sequence:

(SEQ ID NO: 3)
NCCRII_Forward Primer: CGGCCTCGGCCTCCTGTATATA
(SEQ ID NO: 4)
NCCRII_Reverse Primer: TCCACTTCCCCTTGTGCTTTGT

### Reaction Components:

| Component | Stock Conc. | Volume added | | Final conc. | |
|---|---|---|---|---|---|
| Roche LC480 | 2X | 15 | μL | 1X | |
| HRM Master Mix | | | | | |
| MgCl₂ | 25 mM | 4.1 | μL | 3.4 | mM |
| NCCR1_FP | 20 μM | 0.45 | μL | 300 | nM |
| NCCR1_RP | 20 μM | 0.45 | μL | 300 | nM |
| DNA Template | | 10 | μL | | |
| | | 30 | μL total | | |

Master Mix, MgCl₂, and H₂O come as part of Cat #04 909 631 001, Roche.

| | Target | Acquisition | Hold | Ramp Rate | Acquisitions | |
|---|---|---|---|---|---|---|
| Program | (° C.) | Mode | (hh:mm:ss) | (° C./s) | (per ° C.) | Cycles |
| Pre-incubation | 95 | None | 0:10:00 | 4.4 | — | — |
| Amplification | 95 | None | 0:00:10 | 4.4 | — | 50 |
| | 63 | None | 0:00:15 | 2.2 | — | |
| | 72 | Single | 0:00:09 | 4.4 | — | |
| Melting | 95 | None | 0:01:00 | 4.4 | — | — |
| | 40 | None | 0:01:00 | 2.2 | — | |
| | 65 | None | 0:00:01 | 1 | — | |
| | 95 | Continuous | — | 0.02 | 25 | |
| Cooldown | 40 | None | 0:00:10 | 2.2 | — | — |

A second primer set (SEQ ID NO:3 and SEQ ID NO:4) was used to reduce the interference of amplification from genomic DNA in samples with large amounts of gDNA. The second primer set is directed to the same binding site as the first primer set (SEQ ID NO:1 and SEQ ID NO:2), and each primer of the second set is about 3 nucleotides longer than the primers of the first set. Table 5 addresses the relative sensitivity of the two primer sets at low JCV loads. This was tested by spiking whole Mad-4 strain virus (ATCC) into pooled plasma (Bioreclamation) at the concentrations shown. The several replicates at each viral load were then extracted using the MinElute Virus Kit (Qiagen) with slight protocol modifications and run using one half of the eluate for one primer set and the other half of the eluate for the other primer set. Sensitivity was assessed by observing the proportion of positive replicates, meaning that the NCCR_II_{—}1 primer set showed slightly better sensitivity. In these samples, there was a small amount of endogenous JCV in the background; however, this JCV was of the archetype genotype, and thus could readily be distinguished from the spiked JCV and discounted from the analysis.

TABLE 5

| | Total extr. | NCCR1 (old) | NCCR_II_1 (new) |
|---|---|---|---|
| Spike | replicates | total pos. | total pos. |
| 100 cp/mL | 8 | 7** | 8 |
| 50 cp/mL | 12 | 11* ** | 12 |
| 20 cp/mL | 12 | 7 | 6 |
| 10 cp/mL | 12 | 2 | 6 |
| 0 cp/mL | 4 | 0 | 0 |

Plasma used for dilution showed some endogenous JCV that was not included in the table.
*one low positive
**plus some genomic amp. in one replicate

Table 6 addresses the propensity of each primer set to experience interference from genomic DNA. Whole 96-well plates (one plate per primer set) were loaded with reactions containing a level of gDNA commonly seen in extracted plasma samples, and run (up to the 50 amplification cycles) using the gradient feature on the MJ Research Tetrad thermal cycler with the indicated gradients as the annealing step. Immediately upon concluding the 50 cycles, the plates were transferred to the LightCycler 480 for the HRM program and the cooldown program. Positivity of genomic interference was assessed by the proportion of replicates at each annealing temperature that were considered positive. Positivity of replicates was assessed by initial fluorescence values in the HRM program. The NCCR_II_{—}1 primers show a significant reduction in genomic interference. The gradients differ by 5 C due to the difference in calculated Tm between the primer sets (also 5 C).

TABLE 6

| NCCR1 (old) | | | NCCR_II_1 (new) | | |
|---|---|---|---|---|---|
| Temp. | Pos. | Total | | Pos. | Total |
| (C.) | replicate | replicates | Temp. (C.) | replicate | replicates |
| 57.0 | 8 | 8 | 62.0 | 0 | 8 |
| 57.2 | 8 | 8 | 62.2 | 1 | 8 |
| 57.8 | 8 | 8 | 62.8 | 0 | 8 |
| 58.5 | 8 | 8 | 63.5 | 0 | 8 |
| 59.6 | 7 | 7 | 64.6 | 0 | 7 |
| 60.9 | 2 | 8 | 65.9 | 0 | 8 |
| 62.4 | 1 | 8 | 67.4 | 0 | 8 |
| 63.9 | 2 | 8 | 68.9 | 0 | 8 |
| 64.7 | 1 | 8 | 69.7 | 1 | 8 |
| 65.4 | 0 | 8 | 70.4 | 0 | 8 |
| 65.8 | 0 | 8 | 70.8 | 0 | 8 |
| 66.0 | 0 | 8 | 71.0 | 0 | 8 |

### EQUIVALENTS

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

The contents of all references, patents and published patent applications cited throughout this application are incorporated herein by reference in their entirety, particularly for the use or subject matter referenced herein.

What is claimed is:

1. A method for determining the presence of a JC virus mutant in a sample, the method comprising:
amplifying nucleic acid of a JC virus in a sample in the presence of a dye that preferentially binds double-stranded nucleic acid over single stranded nucleic acid,
changing the temperature of the sample and monitoring a signal corresponding to the dye binding to double-stranded nucleic acid to determine the melting temperature of double-stranded nucleic acid in the sample, and
identifying the sample as comprising a JC virus mutant if the melting temperature observed for the sample is different from the melting temperature of a control sample comprising double-stranded JC virus nucleic acid of a non-mutant JC virus.

2. The method of claim 1, wherein amplifying comprises contacting the sample comprising nucleic acid of a JC virus with at least two primers that can hybridize to the nucleic acid of a JC virus.

3. A method for determining the presence of a JC virus mutant in a sample, the method comprising:
obtaining a sample comprising double-stranded JC virus nucleic acid and a dye that preferentially binds double-stranded nucleic acid over single stranded nucleic acid,
changing the temperature of the sample and monitoring a signal corresponding to the dye binding to double-stranded nucleic acid to determine the melting temperature of double-stranded nucleic acid in the sample, and
identifying the sample as comprising a JC virus mutant if the melting temperature observed for the sample is different from the melting temperature of a control sample comprising double-stranded JC virus nucleic acid of a non-mutant JC virus.

4. The method of claim 3, wherein the double-stranded JC virus nucleic acid is amplified.

5. The method of any one of the preceding claims, wherein changing the temperature of the sample comprises heating the sample.

6. The method of any one of the preceding claims, wherein the sample is a biological sample.

7. The method of any one of the preceding claims, wherein the sample is from a subject suspected of being infected with JC virus.

8. The method of any one of the preceding claims, wherein the sample is a blood sample.

9. The method of any one of the preceding claims, wherein the sample is a Cerebrospinal Fluid (CSF) sample.

10. The method of any one of the preceding claims, wherein the sample is a urine sample.

11. The method of any one of the preceding claims, wherein less than 10,000 copies of the JC virus are present in the sample.

12. The method of any one of the preceding claims, wherein less than 1,000 copies of the JC virus are present in the sample.

13. The method of any one of the preceding claims, wherein less than 100 copies of the JC virus are present in the sample.

14. The method of any one of the preceding claims, wherein the double-stranded JC virus nucleic acid comprises the NCCR (Noncoding Control Region) of the JC virus.

15. The method of any one of the preceding claims, wherein the double-stranded JC virus nucleic acid is a part of the NCCR (Noncoding Control Region) of the JC virus.

16. The method of any one of the preceding claims, wherein the double-stranded JC virus nucleic acid is at least 50 nucleotides in length.

17. The method of any one of the preceding claims, wherein the double-stranded JC virus nucleic acid is at least 100 nucleotides in length.

18. The method of any one of the preceding claims, wherein the double-stranded JC virus nucleic acid is at least 500 nucleotides in length.

19. The method of any one of the preceding claims, wherein the mutation includes less than 20 nucleotides.

20. The method of any one of the preceding claims, wherein the mutation includes less than 10 nucleotides.

21. The method of any one of the preceding claims, wherein the mutation is a single base pair mutation.

22. The method of any one of the preceding claims, wherein the primers are nucleic acid primers.

23. The method of any one of the preceding claims, wherein the primers have a sequence comprising SEQ ID NO:1 and SEQ ID NO:2.

24. The method of any one of the preceding claims, wherein the dye is an intercalating dye.

25. The method of any one of the preceding claims, wherein the dye is a fluorescent dye.

26. The method of any one of the preceding claims, wherein the dye is SYBR Green I.

27. The method of any of the preceding claims, wherein the sample is from a subject, and wherein if the sample is identified as comprising a JC virus mutant, the subject is identified as being at risk for developing progressive multifocal leukoencephalopathy (PML).

28. The method of any of the preceding claims, wherein the sample is from a subject, and wherein if the sample is identified as comprising a JC virus mutant, the subject is identified as being inappropriate for treatment comprising immunosuppressants.

29. The method of any of the preceding claims, wherein the sample is from a subject, and wherein if the sample is identified as comprising a JC virus mutant, and the subject is receiving treatment comprising immunosuppressants, the subject is identified as requiring adjustment or termination of treatment comprising immunosuppressants.

30. The method of claim 28 or 29, wherein treatment comprising immunosuppressants comprises treatment including natalizumab.

31. A kit comprising an intercalating fluorescent dye and double-stranded JC virus nucleic acid of a non-mutant JC virus.

32. The kit of claim 31, further comprising a first nucleic acid primer having a sequence comprising SEQ ID NO:1 and a second nucleic acid primer having a sequence comprising SEQ ID NO:2.

33. A nucleic acid primer having a sequence comprising SEQ ID NO:1.

34. A nucleic acid primer having a sequence comprising SEQ ID NO:2.
